(19) United States
(12) Patent Application Publication
(10) Pub. No.: US 2016/0051714 A1
(43) Pub. Date: Feb. 25, 2016

(54) APPARATUS FOR FUMIGATING WITH CHLORINE PEROXIDE GAS AND METHOD THEREFOR

(71) Applicant: PURGOFARM CO., LTD., Yongin (KR)

(72) Inventors: JongRak Kim, Yongin (KR); HeeSeok Park, Gunpo (KR)

(73) Assignee: PURGOFARM CO., LTD., Yongin (KR)

(21) Appl. No.: 14/779,569

(22) PCT Filed: Mar. 18, 2014
(86) PCT No.: PCT/KR2014/002262

§ 371 Date: Sep. 24, 2015

(30) Foreign Application Priority Data
Mar. 25, 2013 (KR) 10-2013-0031274

(51) Int. Cl. A61L 2/20; C25B 1/26; A23B 9/18; A23B 4/16; A23B 7/144

(52) U.S. Cl. CPC A61L 2/20; A23B 4/16; A23B 7/144; A23B 9/18; C25B 1/26

(57) ABSTRACT

Disclosed is an apparatus and a method for fumigation using chlorine dioxide. The apparatus for fumigation using chlorine dioxide includes: a supply part configured to supply the chlorine dioxide; a gas sensor configured to sense the chlorine dioxide in a target space into which mixed gas including the chlorine dioxide is injected; an introduction part configured to introduce dilution gas for diluting the chlorine dioxide; a mixing part connected to the supply part and the introduction part, and configured to generate the mixed gas by mixing the dilution gas with the chlorine dioxide according to information on a concentration of the chlorine dioxide output from the gas sensor so that the concentration of the chlorine dioxide in the target space may be located within a preset actual concentration band; and a transfer part connected to the mixing part to transfer the mixed gas to the target space.

### TECHNICAL FIELD

The present invention relates to an apparatus and a method for fumigation using chlorine dioxide.

### BACKGROUND

When agriculture and livestock products are provided to consumers through the process of storage and distribution, spoilage bacteria, physiological effects, or a temperature condition may be complexly operated in the agriculture and livestock products so that the agriculture and livestock products may be spoiled or the value as a product may be damaged. Since a loss rate has the range of 20% to 30% based on entire agriculture and livestock products, the economic loss range is up to several trillion won.

In order to reduce the loss during the storage and the distribution of the agriculture and livestock products, a cold chain method has been widely used until now. In addition, a controlled atmosphere (CA) storage method and a modified atmosphere (MA) storage method that reduce oxygen and increase carbon dioxide by changing an air composition component of a closed storage space also have been used. Further, recently, a method that adjusts ethylene operating as an ageing hormone of a plant has been used. In the case of livestock products, there is a technology of maintaining freshness by applying various methods such as temperature adjustment and vacuum packing.

However, unless a concentration of a microorganism attached to a surface of agriculture and livestock products is decreased, it is difficult to control the spoilage acceleration of the agriculture and livestock products caused by the microorganism. Sometimes, the microorganism may be removed by washing agriculture products using an antiseptic solution depending on crops. However, most fruit crops including strawberry, grape, and peach and medicinal herbs are basically impossible to make contact with a water solution, and, also, there is no alternative in the case of livestock products.

Even if the contact of the solution is permitted, experimental results report that the gas sterilization effect is much higher than the effect of washing sterilization using the solution when the sterilization is accomplished for the microorganism living in a cracked or damaged gap region of agriculture and livestock products.

In comparison with the solution washing sterilization, when a gas fumigation method is applied, it is possible for gas to make contact with crops through an extremely tiny air hole after a product is packed, so that the sterilization effect may be achieved.

There are various materials used in the above gas fumigation method. In the case of a sulfur fumigating method, there is a problem in that it may be harmful to a human body. In the case of methyl bromide (MB) fumigation, there is a problem in that it may be prohibited to be used as an ozone destruction material. In the meantime, chlorine dioxide is a food additive registered in the Ministry of Food and Drug Safety, and is an organic environmental-friendly material registered in the Ministry for Food, Agriculture, Forestry and Fisheries for the purpose of washing and disinfection a surface of a food.

The chlorine dioxide is an oxide germicide widely used to sterilize bacteria and virus as well as mold. It is well known that a microorganism sterilizing mechanism kills the microorganism by penetrating into a protective film of the microorganism to prevent an enzyme action of a cell due to an oxidizing power.

Since the above sterilizing power of the chlorine dioxide is valid in a wide pH range and is higher at least 2.5 times than the sterilizing pow125er of the chlorine and does not generate a carcinogen such as trihalomethane (THM), the chlorine dioxide is getting the spotlight as an environment-friendly green germicide in Europe or United States of America. The World Health Organization (WHO) and the Food and Agriculture Organization (FAO) of United Nations recommend the chlorine dioxide as the germicide.

The Korean patent laying-open number 10-2012-0092056 of the Applicant discloses an apparatus for generating chlorine dioxide to uniformly maintain a concentration of the chlorine dioxide. Since the concentration of the chlorine dioxide should be changed according to a target space or types of agriculture and livestock products in order to increase the sterilizing effect using the chlorine dioxide, research has been performed to change the concentration of the chlorine dioxide according to the target space or the types of agriculture and livestock products.

### SUMMARY

The present invention may provide an apparatus and a method for fumigation using chlorine dioxide to control a concentration of chlorine dioxide by taking a volume of a target space into consideration.

In accordance with an aspect of the present invention, an apparatus for fumigation using chlorine dioxide includes: a supply part configured to supply the chlorine dioxide; a gas sensor configured to sense the chlorine dioxide in a target space into which mixed gas including the chlorine dioxide is injected; an introduction part configured to introduce dilution gas for diluting the chlorine dioxide; a mixing part connected to the supply part and the introduction part, and configured to generate the mixed gas by mixing the dilution gas with the chlorine dioxide according to information on a concentration of the chlorine dioxide output from the gas sensor so that the concentration of the chlorine dioxide in the target space may be located within a preset actual concentration band; and a transfer part connected to the mixing part to transfer the mixed gas to the target space.

The apparatus for fumigation using chlorine dioxide further includes a controller configured to receive the information from the gas sensor and to output a control signal for adjusting at least one of a flow rate of the dilution gas or a flow rate of the chlorine dioxide so that the concentration of the chlorine dioxide in the target space may be located within the actual concentration band.

The supply part includes: a body including one side in which an electrolyte injection hole into which sodium chlorite is injected and a chlorine dioxide exhaust hole to which the chlorine dioxide is exhausted are formed and the other side, opposite to the one side, in which a surplus gas exhaust hole is formed; a conductive film provided inside the body; at least one positive electrode layer which is able to be connected to a current source, and which makes contact with one side of the conductive film; and a negative electrode layer which is able to be connected to the current source, and which makes contact with the other side of the conductive film, opposite to the one side of the conductive film.

The supply part generates the chlorine dioxide in a gaseous state by passing air bubble through a solvent in which the chlorine dioxide is dissolved.

The mixing part has a volume smaller than an internal volume of the target space, and a maximum concentration and a minimum concentration of the chlorine dioxide in the mixing part is higher than an upper limit concentration and a lower limit concentration of the actual concentration band, respectively.

The target space is a closed space blocked from an outside of the target space, the introduction part dilutes the chlorine dioxide by introducing an internal air of the target space.

A lower concentration of the actual concentration band is 5 ppm or more, and an upper limit concentration is 300 ppm or less.

The apparatus for fumigation using chlorine dioxide further includes an air curtain which is connected to the transfer part, and which is installed close to an opening that is formed in the target space and that is able to communicate with the outside of the target space, and which injects the mixed gas into the inside of the target space.

The apparatus for fumigation using chlorine dioxide further includes a fumigation injecting part which is connected to the transfer part, and which is installed on a ceiling of the target space, and which is configured to inject the mixed gas into the target space through a plurality of injecting holes which are spaced apart from each other.

A reference concentration is set within a preset acceptable concentration band according to a target exposed to the chlorine dioxide in the target space, an upper limit concentration of the actual concentration band is set according to the reference concentration and an upper limit offset, a lower limit concentration of the actual concentration band is set according to the reference concentration and a lower limit offset, and the upper limit concentration and the lower limit concentration are a value located within the acceptable concentration band.

A concentration of the chlorine dioxide in the target space is located within the actual concentration band, and is increased and decreased repeatedly for an exposure time when a target is exposed to the chlorine dioxide.

A target exposed to the chlorine dioxide is exposed to a next actual concentration band which is lower than the actual concentration band, after being exposed to the chlorine dioxide of the actual concentration band in the target space.

The mixed gas having an initial concentration is firstly provided to the target space, and the mixed gas is supplied into the target space during a time between an initial providing time of the mixed gas and an exhaust time of the mixed gas, at least one time.

A maximum concentration and a minimum concentration of the chlorine dioxide of the mixed gas before the mixed gas is supplied into the target space is greater than an upper limit concentration and a lower limit concentration of the actual concentration band, respectively.

A reference concentration is set in a preset acceptable concentration band according to a target exposed to the chlorine dioxide in the target space, the target is exposed to the chlorine dioxide of a next actual concentration band which is lower than the actual concentration band, after the target is exposed to the chlorine dioxide of the actual concentration band in the target space, and the reference concentration is able to be changed according to the target in a case of the actual concentration band and the next actual concentration band is applied regardless of the target.

In accordance with another aspect of the present invention, a method for fumigation using chlorine dioxide includes: supplying chlorine dioxide; sensing the chlorine dioxide in a target space; generating mixed gas by mixing dilution gas with the supplied chlorine dioxide according to information on a concentration of the sensed chlorine dioxide so that the concentration of the chlorine dioxide in the target space may be located within a preset actual concentration band; and transferring the mixed gas to the target space.

At least one of a flow rate of the dilution gas or a flow rate of the chlorine dioxide is adjusted according to the information so that the concentration of the chlorine dioxide in the target space may be located within the actual concentration band.

A lower limit concentration of the actual concentration band is 5 ppm or more and an upper limit concentration is 300 ppm or less.

A reference concentration is set within a preset acceptable concentration band according to a target exposed to the chlorine dioxide in the target space, an upper limit concentration of the actual concentration band is set according to the reference concentration and an upper limit offset, a lower limit concentration of the actual concentration band is set according to the reference concentration and a lower limit offset, and the upper limit concentration and the lower limit concentration are a value located within the acceptable concentration band.

A concentration of the chlorine dioxide in the target space is located within the actual concentration band, and is increased and decreased repeatedly for an exposure time when a target is exposed to the chlorine dioxide.

A target exposed to the chlorine dioxide is exposed to a next actual concentration band which is lower than the actual concentration band, after being exposed to the chlorine dioxide of the actual concentration band.

The target space is a closed space blocked from an outside of the target space.

The mixed gas having an initial concentration is firstly provided to the target space, and the mixed gas is supplied into the target space during a time between an initial providing time of the mixed gas and an exhaust time of the mixed gas, at least one time.

A maximum concentration and a minimum concentration of the chlorine dioxide of the mixed gas before the mixed gas is supplied into the target space is greater than an upper limit concentration and a lower limit concentration of the actual concentration band, respectively.

A reference concentration is set in a preset acceptable concentration band according to a target exposed to the chlorine dioxide in the target space, the target is exposed to the chlorine dioxide of a next actual concentration band which is lower than the actual concentration band, after the target is exposed to the chlorine dioxide of the actual concentration band in the target space, and the reference concentration is able to be changed according to the target in a case of the actual concentration band and the next actual concentration band is applied regardless of the target.

Accordingly, the apparatus and the method for fumigation using chlorine dioxide according to an embodiment of the present invention can control a concentration of chlorine dioxide by taking a volume of a target space into consideration as the chlorine dioxide of the target space satisfies an actual concentration band.

### BRIEF DESCRIPTION OF THE DRAWINGS

The objects, features and advantages of the present invention will be more apparent from the following detailed description in conjunction with the accompanying drawings, in which:

FIG. 1 is a block diagram illustrating an apparatus for fumigation using chlorine dioxide according to an embodiment of the present invention;

FIG. 2 and FIG. 4 are views illustrating a supply part of an apparatus for fumigation using chlorine dioxide according to an embodiment of the present invention;

FIG. 3 is a view illustrating a comparison example of the supply part of an apparatus for fumigation using chlorine dioxide according to an embodiment of the present invention;

FIG. 5 is a graph illustrating an example of an actual concentration band;

FIG. 6 is a graph illustrating a comparison example of concentration control of chlorine dioxide;

FIG. 7 is a graph illustrating an example of concentration control of chlorine dioxide by the apparatus for fumigation using chlorine dioxide according to an embodiment of the present invention;

FIG. 8 is a graph illustrating concentrations of chlorine dioxide in a mixing part and a target space respectively;

FIG. 9 and FIG. 10 are views illustrating modified examples of the apparatus for fumigation using chlorine dioxide according to an embodiment of the present invention;

FIG. 11 to FIG. 13 are views illustrating an acceptable concentration band, a reference concentration, and an actual concentration band;

FIG. 14 is a graph illustrating a reference concentration, an exposure time, and an exposure amount;

FIG. 15 to FIG. 17 are views illustrating an effect according to the use of different actual concentration band with respect to the same target; and

FIG. 18 is a flowchart illustrating a method for fumigation using chlorine dioxide according to an embodiment of the present invention.

### DETAILED DESCRIPTION

Embodiments of the present invention are described with reference to the accompanying drawings in detail. The same reference numbers are used throughout the drawings to refer to the same or like parts. Detailed descriptions of well-known functions and structures incorporated herein may be omitted to avoid obscuring the subject matter of the present invention.

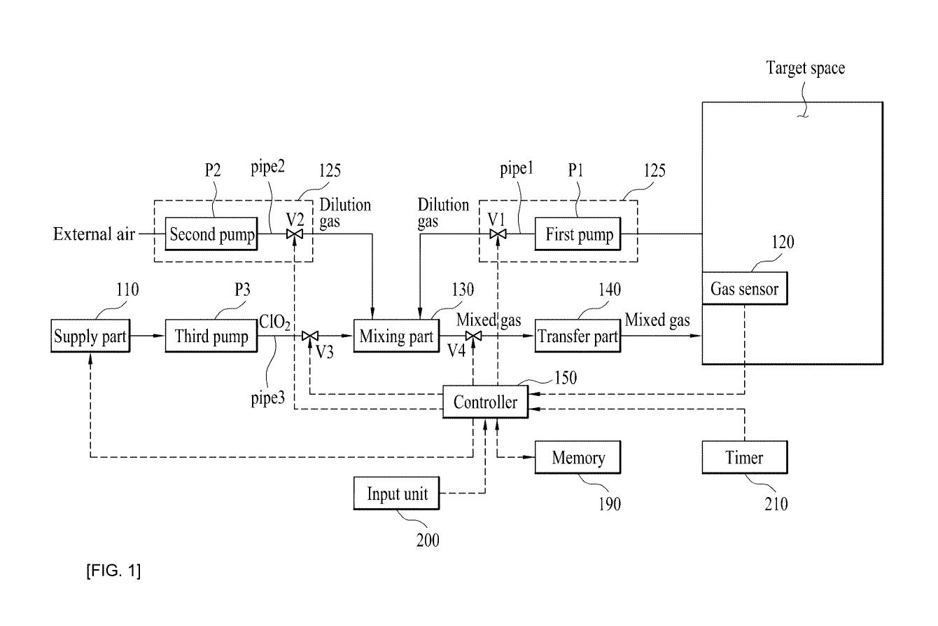

FIG. 1 is a block diagram illustrating an apparatus for fumigationusing chlorine dioxide according to an embodiment of the present invention. As shown in FIG. 1, the apparatus for fumigation using chlorine dioxide according to an embodiment of the present invention may include a supply part 110, a gas sensor 120, an introduction part 125, a mixing part 130, and a transfer part 140.

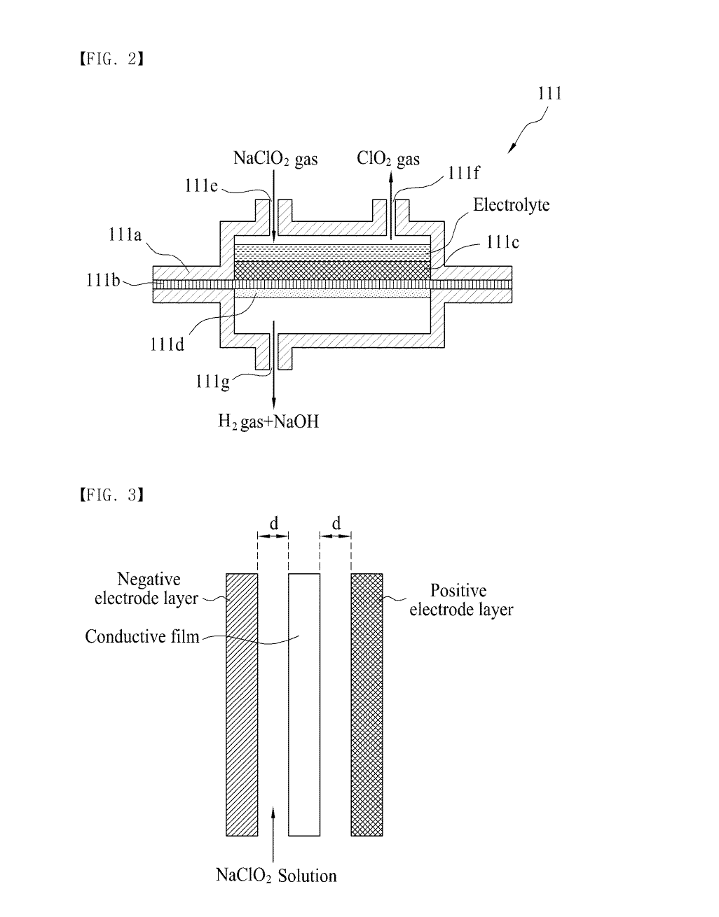

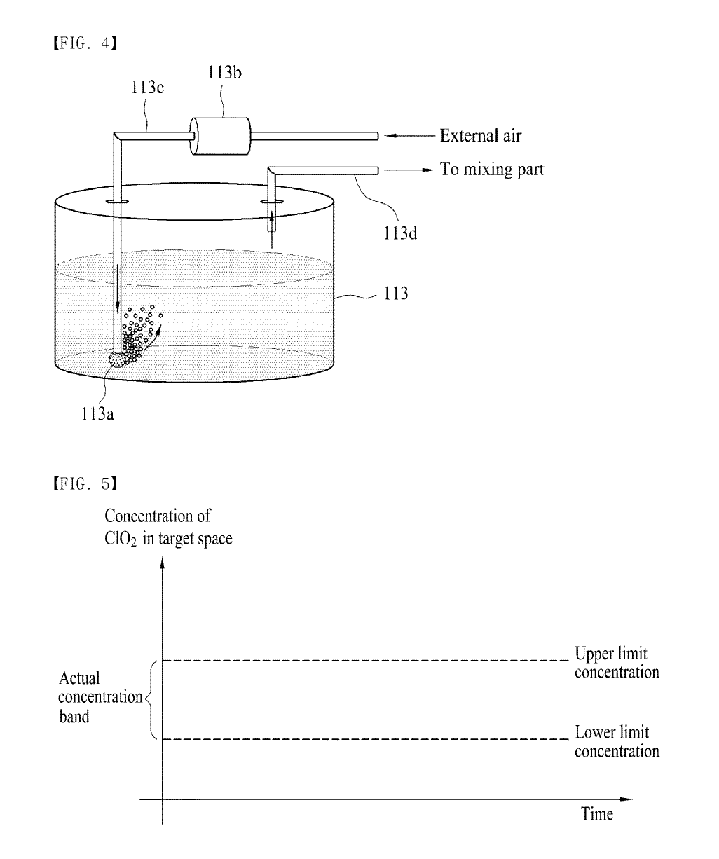

The supply part 110 may supply chlorine dioxide. FIG. 2 and FIG. 3 illustrate the supply part 110 of the apparatus for fumigation using chlorine dioxide according to an embodiment of the present invention. The supply part 110 of FIG. 2 may include an electrolysis device 111. The supply part 110 of FIG. 3 may include a tank 113 for storing a solvent in which the chlorine dioxide is dissolved.

As shown in FIG. 2, the supply part 110 may include the electrolysis device 111. The electrolysis device 111 may include a body 111a, a conductive film 111b, a positive electrode layer 111c, and a negative electrode layer 111d. The body 111a may include one side in which an electrolyte injection hole 111e into which sodium chlorite NaClO2 is injected and a chlorine dioxide exhaust hole 111f to which the chlorine dioxide is exhausted are formed and the other side in which a surplus gas exhaust hole 111g is formed. The conductive film 111b may be provided inside the body 111a. At least one positive electrode layer 111c may be connected to a current source and may make contact with one side of the conductive film 111b. The negative electrode layer 111d may be connected to the current source and may make contact with the other side of the conductive film 111b opposite to the one side.

The conductive film 111b may be formed of a proton conductive material, and may be provided with hydrocarbon and fluorocarbon resin. Here, the fluorocarbon resin may have excellent oxidation resistance with respect to halogen, strong acid, and base. The positive electrode layer 111c may cause oxidation reaction with the electrolyte sodium chlorite. The negative electrode layer 111d may cause reduction reaction.

The positive electrode layer 111c and the negative electrode layer 111d may further include an electrochemical catalyst in order maximize an efficiency of oxidation-reduction reaction. The electrochemical catalyst may include platinum, palladium, rhodium, iridium, ruthenium, osmium, carbon, silver, tantalum, tin, indium, nickel, tungsten, manganese, and a mixture, an oxide, and an alloy including at least one of these elements, or a combination thereof.

The sodium chlorite NaClO2 injected through the electrolyte injection hole 111e may include sodium chlorite NaClO2 salt and water H2O. The sodium chlorite NaClO2 salt may be converted into chlorine dioxide ClO2 gas, a sodium ion Na+, and electron e by oxidation reaction of the positive electrode layer 111c. The water may be converted into oxygen O2 gas, a hydrogen ion H+, and electron e by oxidation reaction of the positive electrode layer 111c.

The chlorine dioxide ClO2 gas and the oxygen O2 gas generated by the oxidation reaction of the positive electrode layer 111c may be exhausted to an outside through the gas exhaust hole 112. The sodium ion Na+ and the hydrogen ion H+ may be transferred to the negative electrode layer 111d by passing through the conductive film 111b due to electric attraction.

In this case, the water H2O may be transferred together with the sodium ion Na+ and the hydrogen ion H+. The hydrogen ion H+ transferred to the negative electrode layer 111d may be transformed into hydrogen due to reduction reaction by the negative electrode layer 111d. The sodium ion Na+ may be bonded to OH— of the water H2O so that sodium hydroxide NaOH may be generated and may be exhausted through the surplus gas exhaust hole 111g.

Since the electrochemical catalyst of the negative electrode layer 111d and the positive electrode layer 111c accelerates the oxidation reaction of the positive electrode layer 111c and the reduction reaction of the negative electrode layer 111d, a gap d, shown in the comparison example of FIG. 3, between the conductive film 111b and the negative electrode layer 111d and the positive electrode layer 111c may be removed.

Since the above gap serves as resistance to interrupt a flow of an electric current, when both sides of the conductive layer 111b make contact with the positive electrode layer 111c and the negative electrode layer 111d according to the embodiment of the present invention, there is no gap between the conductive film 111b and the positive electrode layer 111c, and between the conductive film 111b and the negative electrode layer 111d, thereby reducing power consumption necessary for electrolysis to efficiently obtain chlorine dioxide.

Further, as illustrated in the comparison example of FIG. 3, when the negative electrode layer and the positive electrode layer are spaced apart from the conductive film, in order to prevent deterioration of the efficiency of the electrolysis, sodium chloride NaCl may be provided together with the sodium chlorite. In this case, chlorine may be generated by the electrolysis of the sodium chloride, which may harm a human body.

Meanwhile, in a case of the embodiment of the present invention, since there is no gap between the conductive film 111b and the positive electrode layer 111c and the negative electrode layer 111d, generation efficiency of the chlorine dioxide gas using the electrolysis may be improved. Accordingly, since there is no need to provide the sodium chloride as illustrated in the comparison example, the damage due to the chlorine may be prevented.

Like this, since the positive electrode layer 111c and the negative electrode layer 111d make contact with both sides of the conductive film 111b, the supply part 110 of an apparatus for fumigation using chlorine dioxide according to an embodiment of the present invention may generate the chlorine dioxide through an electrolyte having no sodium chloride.

As described above, the supply part 110 may include the electrolysis device 111 to generate the chlorine dioxide gas. However, unlike this, the supply part 110 may include the tank 113 for storing a solvent in which the chlorine dioxide is dissolved, and the chlorine dioxide gas may be discharged from the tank 113.

That is, as shown in FIG. 4, the supply part 110 of the apparatus for fumigation using chlorine dioxide according to an embodiment of the present invention may include the tank 113. The tank 113 may store the solvent in which the chlorine dioxide is dissolved. According to the embodiment of the present invention, the solvent may be water.

An air bubble generator 113a may be installed at an end portion of an air tube 113c. The air bubble generator 113a may be put in the solvent. A plurality of fine holes may be formed in a surface of the air bubble generator 113a. An air bubble generation pump 113b may make the internal air or external air of a target space run to the air bubble generator 113a through the air tube 113c. The external air may pass through the hole of the air bubble generator 113a to generate air bubble.

When the air bubble is generated, the chlorine dioxide dissolved in the solvent may be gasified and may be transferred to the mixing part 130 through a tank pipe 113d which is connected to the tank 113. In this case, the tank pipe 113d may be connected to a third pump P3 of FIG. 1.

Meanwhile, the gas sensor 120 of FIG. 1 may sense the chlorine dioxide of a target space into which mixed gas including the chlorine dioxide is injected. The target space may be a space in which fumigation is accomplished by using the chlorine dioxide. For example, the target space may be a storehouse, a container box, a hog yard, or a chicken yard in which agriculture and livestock products are kept or grown, but it is not limited thereto.

The introduction part 125 may introduce dilution gas for diluting the chlorine dioxide. In this case, the dilution gas may be the external air of the target space or the internal air of the target space. The concentration of the chlorine dioxide for the mixed gas supplied to the target space may be adjusted through the dilution gas. The method for using the internal air of the target space as the dilution gas is described in detail later.

The mixing part 130 may be connected to the supply part 110 and the introduction part 125. The mixing part 130 may generate mixed gas by mixing the chlorine dioxide with the dilution gas so that the concentration of the chlorine dioxide in the target space may exist within a preset actual concentration band.

The transfer part 140 may be connected to the mixing part 130 and transfer the mixed gas to the target space. According to the embodiment of the present invention, the transfer part 140 may include a pipe or a tube through which the mixed gas flows.

When the mixed gas obtained by mixing the chlorine dioxide with the dilution gas is supplied to the target space, as shown in FIG. 3 of the Korean patent laying-open number 10-2012-0092056, it may be difficult to maintain a constant concentration of the chlorine dioxide.

In order to store or grow the agriculture and livestock products in the target space, a volume of the target space should be large. If the volume of the target space is increased, it may be difficult to uniformly maintain the concentration of the chlorine dioxide with a constant value in a full target space.

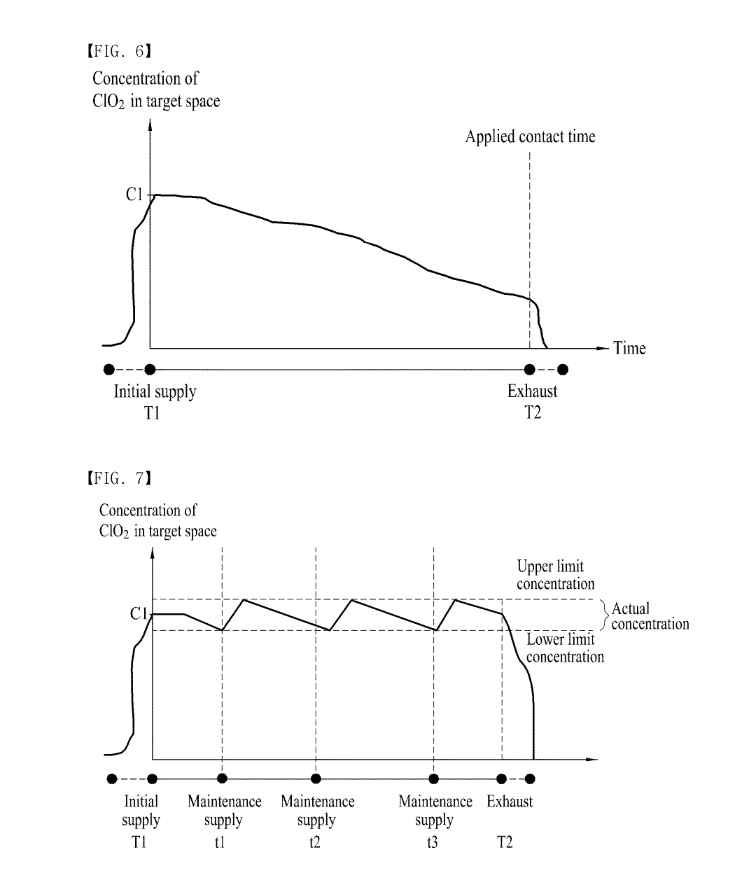

Meanwhile, as shown in FIG. 5, the apparatus for fumigation using chlorine dioxide according to an embodiment of the present invention may adjust the concentration of the chlorine dioxide in the target space so that the concentration of the chlorine dioxide of the target space may exist within a preset actual concentration band.

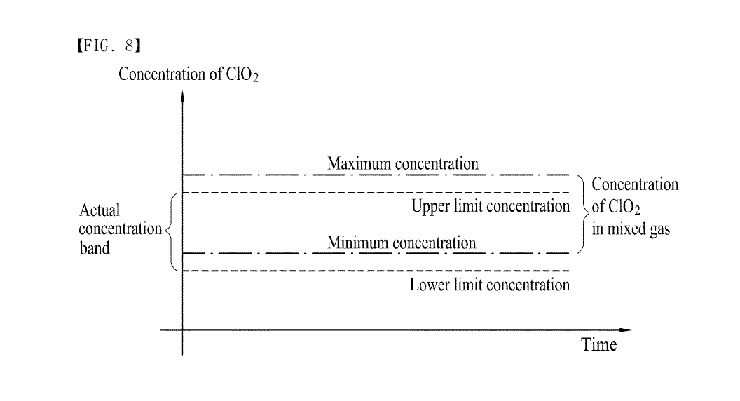

FIG. 6 is a graph illustrating a comparison example of concentration control of chlorine dioxide, and FIG. 7 is a graph illustrating an example of concentration control of chlorine dioxide by the apparatus for fumigation using chlorine dioxide according to an embodiment of the present invention.

As shown in FIG. 6, when the mixed gas of the chlorine dioxide having an initial concentration C1 is initially supplied into the target space, the mixed gas may be spread to the target space so that the concentration of chlorine dioxide may be gradually decreased.

On the other hand, as shown in FIG. 7, the apparatus for fumigation using chlorine dioxide according to an embodiment of the present invention may initially supply the mixed gas of chlorine dioxide having the initial concentration C1, and may supply the mixed gas into the target space at the time between an initial providing time T1 of the mixed gas and an exhaust time T2 of the mixed gas at least one time. In this case, the exhaustion of the mixed gas may be accomplished by exhausting the mixed gas to the outside of the target space.

That is, the mixed gas may be initially supplied into the target space and may be spread into the target space so that the concentration of chlorine dioxide in the target space sensed by the gas sensor 120 may be gradually decreased. The gas sensor 120 may output information on the concentration of chlorine dioxide approaching the lower limit concentration.

As the concentration of the chlorine dioxide approaches the lower limit concentration, the apparatus for fumigation using chlorine dioxide according to an embodiment of the present invention may supply the mixed gas into the target space at a point of time t1. When the mixed gas is supplied into the target space, the mixed gas may be temporarily concentrated in a partial region of the target space, so that the concentration of the chlorine dioxide may be gradually increased.

Next, since the mixed gas is spread to the target space as time goes on, the concentration of the chlorine dioxide may be gradually decreased. In this case, the upper limit concentration and the lower limit concentration of the actual concentration band may be a maximum value at which the concentration of the chlorine dioxide in the target space is increased after providing the mixed gas, and a minimum value at which the concentration of the chlorine dioxide in the target space is decreased, respectively.

The gas sensor 120 may output information on the concentration of the chlorine dioxide approaching the lower limit concentration. The apparatus for fumigation using chlorine dioxide according to an embodiment of the present invention may supply the mixed gas to the target space at the point of time t2. As the mixed gas is repeatedly supplied, the concentration of the chlorine dioxide may be repeatedly increased and decreased in the target space.

As described above, the apparatus for fumigation using chlorine dioxide according to an embodiment of the present invention may adjust the concentration of the chlorine dioxide in the target space to be located between the upper limit concentration and the lower limit concentration of the actual concentration band, thereby easily controlling the concentration of the chlorine dioxide even if the volume of the target space is large.

In order to make the concentration of the chlorine dioxide in the target space to be located within the actual concentration band, the concentration of the chlorine dioxide for the mixed gas may be controlled in various methods.

For example, the concentration of the chlorine dioxide for the mixed gas may be adjusted by controlling a flow rate of the dilution gas, or may be adjusted by controlling a flow rate of the concentration of the chlorine dioxide supplied from the supply part 110.

As shown in FIG. 1, the flow rate of the dilution gas may be controlled by changing an amount of opening and closing of a first valve V1 of the introduction part 125 or by changing the discharge pressure of a first pump P1. That is, the introduction part 125 may include a first dilution gas pipe (pipe 1) connecting the mixing part 130 to the target space, a first valve V1 installed in the first dilution gas pipe (pipe 1), and a first pump P1 configured to make the dilution gas in the target space run to the mixing part 130 through the first dilution gas pipe (pipe 1).

When the dilution gas is the external air of the target space, the flow rate of the dilution gas may be controlled by changing an amount of opening and closing of a second valve V2 of the introduction part 125 or by changing the discharge pressure of a second pump P2. That is, the introduction part 125 may include a second dilution gas pipe (pipe2) through which the external air, as the dilution gas, flows, a second valve V2 installed in the second dilution gas pipe (pipe2), and a second pump P2 configured to make the external air run to the mixing part 130 through the second dilution gas pipe (pipe2).

The flow rate of the chlorine dioxide instead of the dilution gas may be controlled by changing an amount of opening and closing of a third valve V3 installed in a chlorine dioxide pipe (pipe3) connected between the supply part 110 and the mixing part 130, or by changing the discharge pressure of a third pump P3.

Meanwhile, the mixing part 130 of FIG. 1 may provide a space in which the dilution gas is mixed with the chlorine dioxide and may include a fan (not shown) configured to easily perform mixing.

Further, the apparatus for fumigation using chlorine dioxide according to an embodiment of the present invention may include a third pump P3 configured to make the mixed gas run to the target space in a certain direction. Since the third pump P3 is installed in the chlorine dioxide pipe (pipe3) between the supply part 110 and the mixing part 130, the third pump P3 make the mixed gas run to the transfer part 140 and make the chlorine dioxide run to the mixing part 130 from the supply part 110.

A fourth valve V4 may be installed in a flow path through which the mixed gas flows between the mixing part 130 and the transfer part 140. The fourth valve V4 may control the flow rate of the mixed gas supplied to the target space.

The apparatus for fumigation using chlorine dioxide according to an embodiment of the present invention may further include a controller 150. The controller 150 may receive information from the gas sensor 120 and output a control signal to adjust at least one of a flow rate of the dilution gas or a flow rate of the chlorine dioxide so that the concentration of the chlorine dioxide may be located within the actual concentration band.

In FIG. 1, a dotted line connected to the controller 150 may represent information on the concentration of the chlorine dioxide input to the controller 150 and a control signal for controlling the opening/closing of the first valve V1 to the fourth valve V4 and the amount of opening/closing. At least one of the flow rate of the dilution gas or the flow rate of the chlorine dioxide may be adjusted by controlling the first valve V1 to the fourth valve V4. Accordingly, the concentration of the chlorine dioxide in the target space may be controlled so that the concentration of the chlorine dioxide in the target space may be located within the actual concentration band.

Further, when the supply part 110 includes the electrolysis device 111 as shown in FIG. 2, the controller 150 may output a control signal for controlling the amplitude of electric current supplied to the positive electrode layer 111c and the negative electrode layer 111d. Since the method of controlling the amplitude of the electric current supplied to the positive electrode layer 111c and the negative electrode layer 111d of the electrolysis device 111 is a general technology, a detailed description thereof is omitted.

Meanwhile, the mixing part 130 may have a volume smaller than an internal volume of the target space. As shown in FIG. 8, the maximum concentration and the minimum concentration of the chlorine dioxide in the mixing part 130 may be higher than the upper limit concentration and the lower limit concentration of the actual concentration band, respectively.

Since a difference between the concentrations of the chlorine dioxide in a different region of the target space may be increased if the target space is increased, it may be difficult to fill the whole target space with chlorine dioxide having a high concentration.

In order to prevent this, in the apparatus for fumigation using chlorine dioxide according to an embodiment of the present invention, the maximum concentration and the minimum concentration of the chlorine dioxide of the mixed gas before the mixed gas is supplied to the target space may be greater than the upper limit concentration and the lower limit concentration of the actual concentration band, respectively. In this case, when the volume of the internal space of the mixing part 130 is smaller than the volume of the target space, it may be easy to fill the mixing part 130 with the chlorine dioxide of high concentration.

Further, in order to fill the target space with the chlorine dioxide of high concentration and to maintain the actual concentration band of the chlorine dioxide, the introduction of the external air into the target space may be blocked. To this end, the target space may be a closed space blocked from the outside of the target space.

As described above, if the external air is introduced into the target space, it may be difficult to maintain the chlorine dioxide with a high concentration in the target space. Further, when the external air is used as the dilution gas, the external air is introduced into the target space as a part of the mixed gas. Hence, it may be difficult to maintain the chlorine dioxide in the target space with a high concentration.

In order to prevent this, the introduction part 125 of FIG. 1 may dilute the chlorine dioxide by introducing the internal air of the target space which is a closed space. Since the introduction of the external air into the target space is blocked and the air in the target space is used as the dilution gas instead of using the external air, the apparatus for fumigation using chlorine dioxide according to an embodiment of the present invention may easily maintain a desired actual concentration band of the chlorine dioxide in the target space.

In this case, since the mixed gas is supplied into the target space, the air in the target space may include chlorine dioxide. Accordingly, when the air in the target space is used as the dilution gas, the dilution gas may include the chlorine dioxide.

According to an embodiment of the present invention, in order to fill the target space with the chlorine dioxide of high concentration, the lower limit concentration of the actual concentration band may be 5 ppm or more, and the upper limit concentration of the actual concentration band may be 300 ppm or less.

When the chlorine dioxide of 5 ppm or more is provided into the target space, the sterilization for a target such as agriculture and livestock products may be sufficiently accomplished within a short time. In addition, when the chlorine dioxide having a concentration of 300 ppm or less is supplied into the target space, the sterilization for agriculture and livestock products having a large shell thickness such as orange or sweet pumpkin may be sufficiently accomplished within a short time.

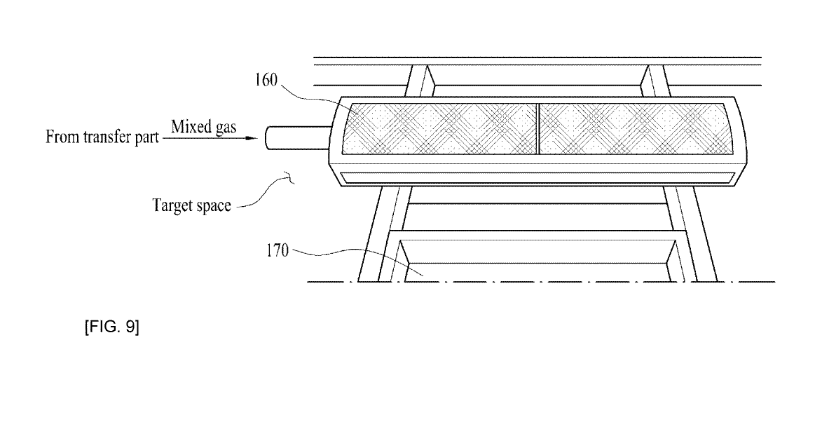

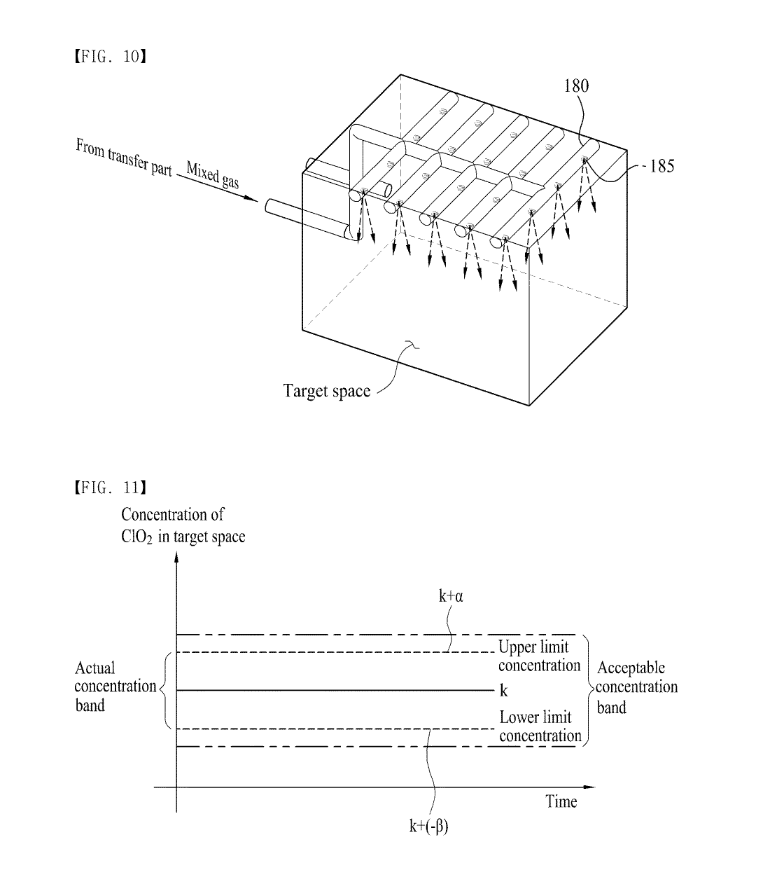

FIG. 9 and FIG. 10 illustrate modified examples of the apparatus for fumigation using chlorine dioxide according to an embodiment of the present invention.

As shown in FIG. 9, the apparatus for fumigation using chlorine dioxide according to an embodiment of the present invention may further include an air curtain 160. The air curtain 160 may be connected to the transfer part 140 and may be installed close to an opening 170 which is formed in the target space and which is able to communicate with the outside of the target space, and may inject the mixed gas into the inside of the target space. In this case, the opening 170 may be a window or a door of the target space, but it is not limited thereto.

When the opening 170 which enables to communicate with the outside of the target space is formed, the target space may be difficult to implement a closed space. Accordingly, although the air curtain 160 injects the chlorine dioxide of high concentration, the target space may not maintain the chlorine dioxide with a high concentration.

Accordingly, the air curtain may be suitable to fill the target space with the chlorine dioxide of low concentration. When the target space is filed with the chlorine dioxide of low concentration, the upper limit concentration of the actual concentration band in the target space may be 0.3 ppm or less and the lower limit concentration of the actual concentration band in the target space may be 0.03 ppm or less. Even if a person is exposed to the chlorine dioxide of 0.3 ppm for about fifteen minutes, the chlorine dioxide is harmless to the human body. In addition, when the concentration of the chlorine dioxide is 0.03 ppm or more, the sterilization for the agriculture and livestock products may be accomplished.

As shown in FIG. 10, the apparatus for fumigation using chlorine dioxide according to an embodiment of the present invention may further include a fumigation injection part 180. The fumigation injection part 180 may be connected to the transfer part 180, may be installed on a ceiling of the target space, and may inject the mixed gas to the target space through a plurality of injection holes 185 which are spaced apart from each other.

Meanwhile, the apparatus for fumigation using chlorine dioxide according to an embodiment of the present invention may store a preset acceptable concentration band according to a target exposed to the chlorine dioxide in the target space. The acceptable concentration band may be stored in a memory 190 of FIG. 1. The controller 150 may access the memory 190 to read the acceptable concentration band according to the target.

FIG. 11 to FIG. 13 are views illustrating an acceptable concentration band, a reference concentration, and an actual concentration band. The acceptable concentration band according to the target stored in the apparatus for fumigation using chlorine dioxide according to an embodiment of the present invention may be obtained through an experiment for the sterilization performance according to the change in the concentration of the chlorine dioxide.

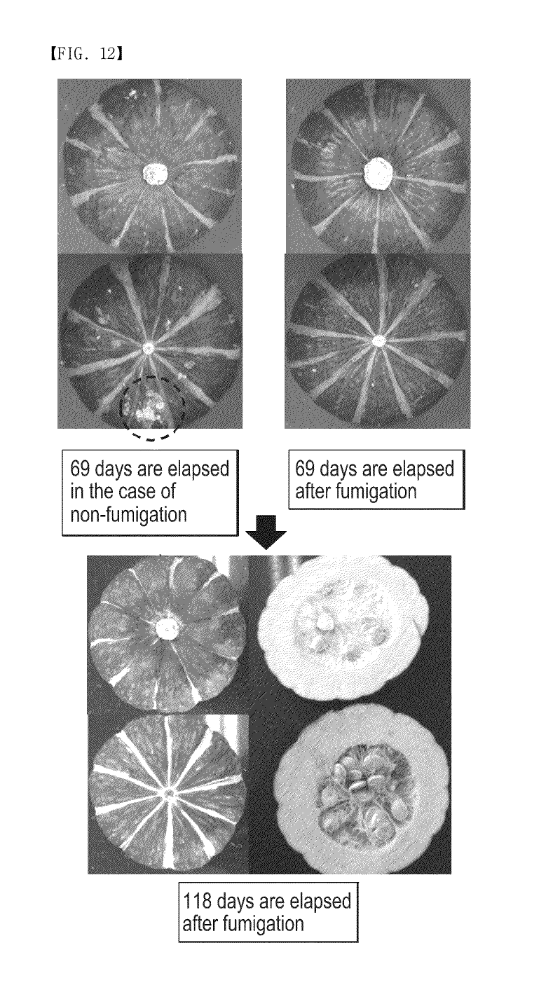

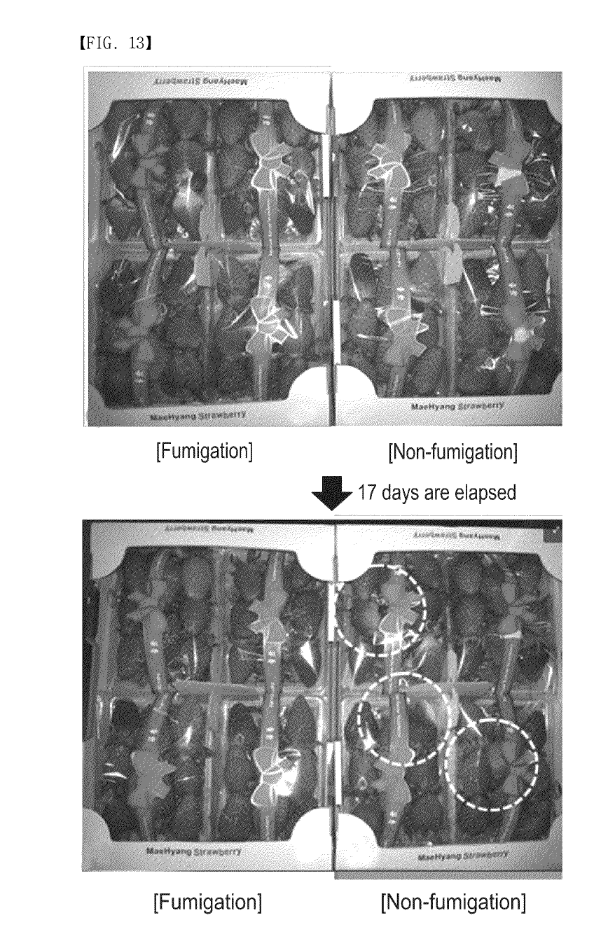

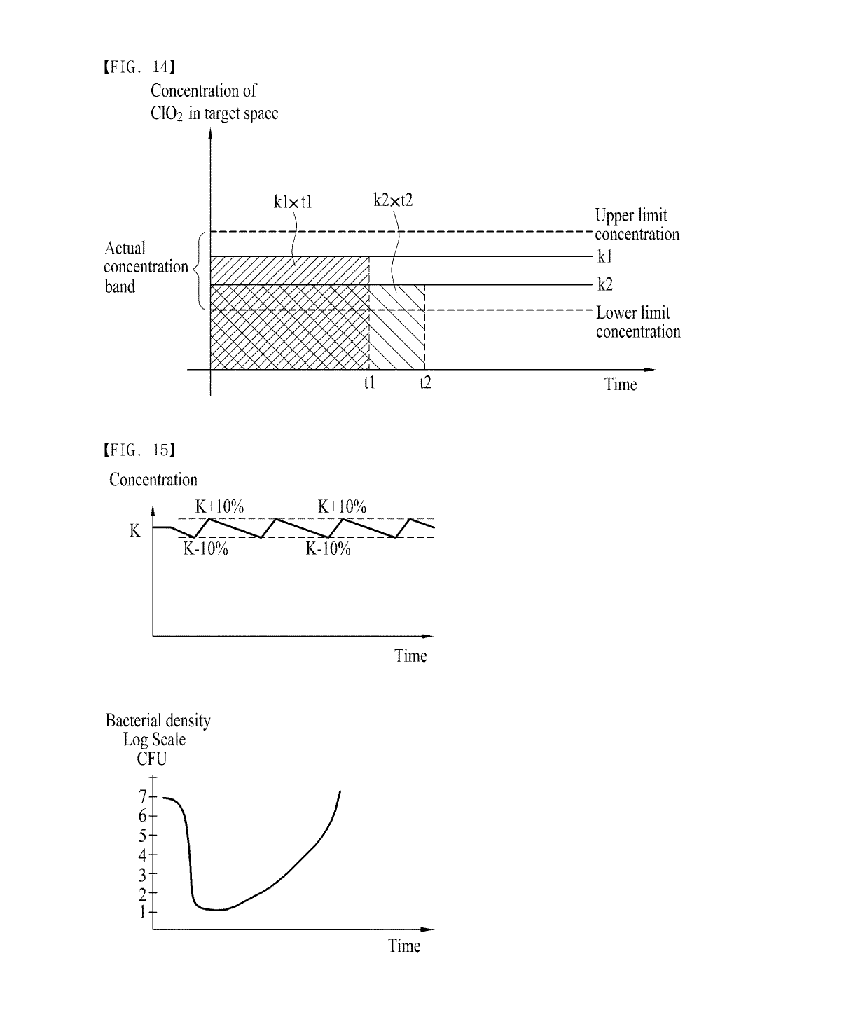

As shown in FIG. 12 and FIG. 13, the sterilization performance may be estimated as a start time of spoilage of the agriculture and livestock products when the agriculture and livestock products are exposed to the chlorine dioxide.

As shown in FIG. 12, since a white spot occurs, as shown in a dotted line circle, in the shell of a pumpkin which is not fumigated when 69 days are elapsed after starting a storing, it can be recognized that the shell of the pumpkin goes sour. On the other hand, even when 118 days are elapsed after starting the storing, the pumpkin fumigated by the apparatus for fumigation using chlorine dioxide according to an embodiment of the present invention does not go sour. Such experiment for the pumpkin was repeatedly performed by changing the concentration of the chlorine dioxide and the acceptable concentration band for the pumpkin may be set through the experimental result.

The same experiment was accomplished for the strawberry.

As shown in FIG. 13, the strawberry which is not fumigated is spoiled as shown in a dotted line circle when 17 days are elapsed after starting the storing. On the other hand, even when 17 days are elapsed after starting the storing, it can be recognized that the strawberry fumigated by the apparatus for fumigationusing chlorine dioxide according to an embodiment of the present invention does not go sour. Such experiment for the strawberry was repeatedly performed by changing the concentration of the chlorine dioxide and the acceptable concentration band for the strawberry may be set through the experimental result.

The acceptable concentration band according to the target obtained through the above experiment may be stored in the memory 190 of FIG. 1.

The embodiment of the present invention describes the acceptable concentration band of the pumpkin and the strawberry, but it is not limited thereto. The acceptable concentration band may be set for various agricultural products such as oranges, grapes, peaches, and paprika as well as the pumpkin and the strawberry or may be set for livestock products such as beef, pork, and chicken.

A reference concentration k may be set within the acceptable concentration band. Since the acceptable concentration band may be changed according to a type of the target, the reference concentration k may also be changed according to the type of the target.

The upper limit concentration of the actual concentration band may be set according to the reference concentration k and an upper limit offset ( ), and the lower limit concentration of the actual concentration band may be set according to the reference concentration k and a lower limit offset (−). For example, the upper limit concentration may be a sum of the reference concentration k and the upper limit offset ( ), and the lower limit concentration may be a sum of the reference concentration k and the lower limit offset (−). The upper limit concentration and the lower limit concentration of the actual concentration band may be a value within the acceptable concentration band. In this case, an absolute value of the upper limit offset ( ) may be identical with or different from an absolute value of the lower limit offset (−).

The reference concentration may also be stored in the memory 190 together with the acceptable concentration band according to the target. On the other hand, in the case in which the acceptable concentration band is stored in the memory 190 and the reference concentration is input to the controller 150 through an input unit 200 by an operator of the apparatus for fumigation using chlorine dioxide, when the input reference concentration exists within the acceptable concentration band, the controller 150 may set the input value as the reference concentration.

The input unit 200 may include an input switch, a key pad, a keyboard, or a touch pad, but it is not limited thereto. Further, the actual concentration band for each target calculated according to the acceptable concentration band, the reference concentration, and the upper limit offset ( ) and the lower limit offset (−) may be stored in the memory 190.

FIG. 14 is a graph illustrating a reference concentration, an exposure time, and an exposure amount. As shown in FIG. 14, the exposure amount may be calculated based on the reference concentrations k1 and k2 and the exposure time when the target is exposed to the chlorine dioxide. In this case, the reference concentration and the exposure time may be changed to satisfy the exposure amount for the target.

For example, different reference concentrations k1 and k2 may be set for the same target A. Since the reference concentration k2 is lower than the reference concentration k1, the exposure time may be increased in the case of the reference concentration k2 in comparison with the case of the reference concentration k1 in order to obtain the same sterilization effect for the target A.

When the reference concentration is k1, the exposure amount of the chlorine dioxide for the target A may be k1×t1. When the reference concentration is k2, the exposure amount of the chlorine dioxide for the target A may be k2×t2. In this case, the exposure amount k1×t1 may be identical with the exposure amount k2×t2.

The exposure amount for the target may be calculated by the controller 150 or the previously calculated exposure amount may be stored in a storage unit.

Referring to FIG. 7, the concentration of the chlorine dioxide in the target space may be within the actual concentration band, and may be repeatedly increased and decreased for the exposure time when the target is exposed to the chlorine dioxide.

FIG. 15 to FIG. 17 are views illustrating an effect according to the use of different actual concentration band with respect to the same target.

FIG. 15 illustrates a bacterial density that exists in a target A when ten days are elapsed after the target A is exposed to the chlorine dioxide satisfying the actual concentration band of high concentration for thirty minutes. In this case, the upper limit offset may be 10% of the reference concentration k, and the lower limit offset may be −10% of the reference concentration k. As shown in FIG. 15, since the target A is exposed to the chlorine dioxide of high concentration, the bacterial density may be rapidly decreased up to two days or three days, but, since then, may be slowly increased.

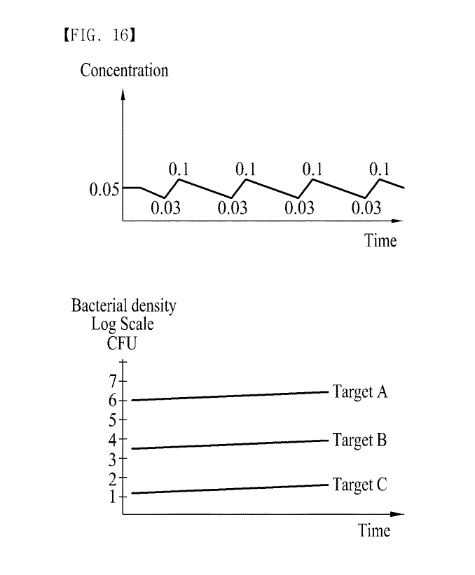

FIG. 16 illustrates the bacterial density that exists in a target A, a target B, and a target C when the target A is exposed to the chlorine dioxide satisfying the actual concentration band of low concentration for ten days. In this case, the upper limit concentration and the lower limit concentration of the actual concentration band may be 0.1 ppm and 0.03 ppm, respectively. Since the target A, the target B, and the target C are exposed to the chlorine dioxide of low concentration, it can be recognized that the bacterial density is not decreased up to 10 days and the change in the bacterial density is small.

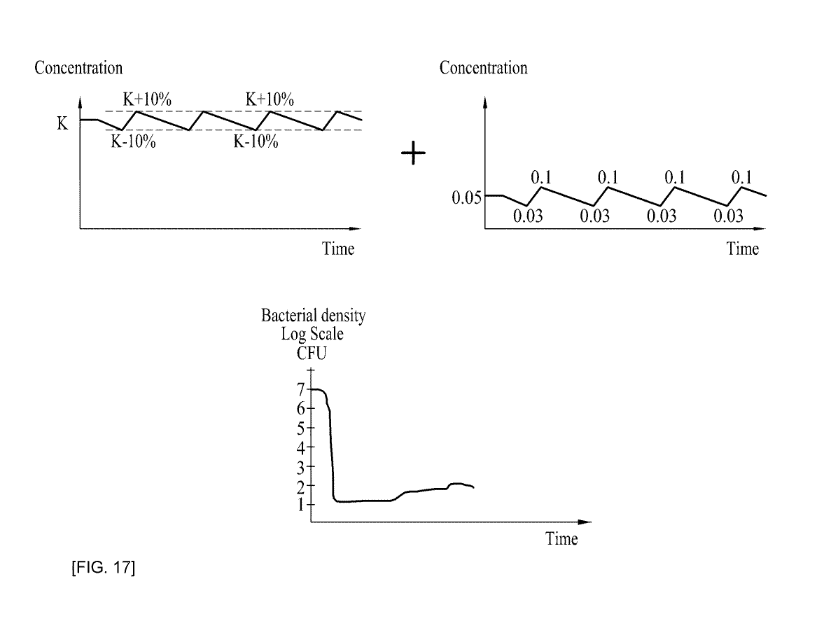

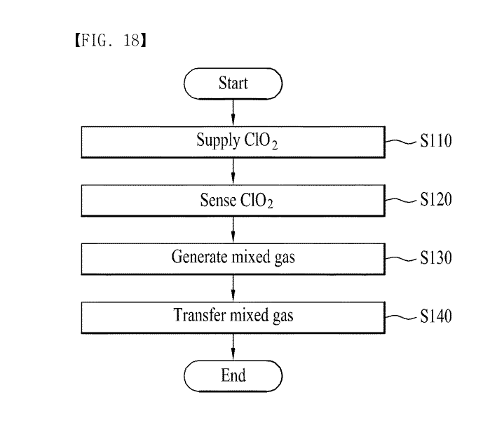

FIG. 17 illustrates the bacterial density that exists in a target A when the target A is exposed to the chlorine dioxide satisfying the actual concentration band of low concentration for ten days after the target A is exposed to the chlorine dioxide satisfying the actual concentration band of high concentration for thirty minutes. In this case, the upper limit offset in the actual concentration band of high concentration may be 10% of the reference concentration k, and the lower limit offset may be −10% of the reference concentration k. Moreover, the upper limit concentration and the lower limit concentration of the actual concentration band of low concentration may be 0.1 ppm and 0.03 ppm, respectively.

As shown in FIG. 7, since the target A is exposed to the chlorine dioxide of high concentration, after the bacterial density is rapidly decreased for two days or three days, it can be recognized that the bacterial density is continuously maintained

As described above, it can be recognized that the highest sterilization effect may be achieved when the target is exposed to the chlorine dioxide of a next actual concentration band which is lower than the actual concentration band after the target is exposed to the chlorine dioxide of the actual concentration band in the target space.

As described above, the target may be exposed to the chlorine dioxide of low concentration satisfying the next actual concentration band, after the target is exposed to the chlorine dioxide of high concentration satisfying the actual concentration band. In this case, in the case of the actual concentration band, the reference concentration may be changed according to the target, but the next actual concentration band may be applied regardless of the target. Since the next actual concentration band corresponds to the chlorine dioxide of low concentration, as mentioned above, the upper limit concentration of the next actual concentration band may be 0.3 ppm or less, and the lower limit concentration of the next actual concentration band may be 0.01 ppm or more.

The target space may be filled with the chlorine dioxide of low concentration satisfying the next actual concentration band, after the target space is filled with the chlorine dioxide of high concentration satisfying the actual concentration band. In order to easily change the concentration of the chlorine dioxide in the target space, the mixed gas in the target space may be exhausted after the chlorine dioxide of the actual concentration band is transferred, and the chlorine dioxide of the next actual concentration band may be transferred.

As the concentration of the chlorine dioxide satisfying the actual concentration band is increased and decreased repeatedly, the concentration of the chlorine dioxide in the target space satisfying the next actual concentration band may also be increased and decreased repeatedly within the next actual concentration band.

Hereinafter, the method for fumigation using chlorine dioxide according to an embodiment of the present invention is described with reference to a drawing.

FIG. 18 is a flowchart illustrating a method for fumigation using chlorine dioxide according to an embodiment of the present invention. As shown in FIG. 18, the method for fumigation using chlorine dioxide according to an embodiment of the present invention may include the steps of: supplying chlorine dioxide (S110), sensing the chlorine dioxide in the target space (S120), generating mixed gas by mixing dilution gas with the supplied chlorine dioxide so that a concentration of the chlorine dioxide in the target space may be located within a preset actual concentration band according to information on a concentration of the sensed chlorine dioxide (S130); and transferring the mixed gas to the target space (S140).

According to such information, at least one of a flow rate of the dilution gas or a flow rate of the chlorine dioxide may be adjusted so that the concentration of the chlorine dioxide in the target space may be located within the actual concentration band.

The lower limit concentration of the actual concentration band may be 5 ppm or more and the upper limit concentration may be 300 ppm or less.

The reference concentration may be set within a preset acceptable concentration band according to the target exposed to the chlorine dioxide in the target space. The upper limit concentration of the actual concentration band may be set according to the reference concentration and the upper limit offset. The lower limit concentration of the actual concentration band may be set according to the reference concentration and the lower limit offset. The upper limit concentration and the lower limit concentration may be a value that exists within the acceptable concentration band.

The exposure amount may be calculated based on the reference concentration and the exposure time when the target is exposed to the chlorine dioxide.

The reference concentration and the exposure time may be changed to satisfy the exposure amount for the target.

The concentration of the chlorine dioxide in the target space may be located within the actual concentration band, and may be repeatedly increased and decreased during the exposure time when the target is exposed to the chlorine dioxide.

The target may be exposed to the chlorine dioxide of the next actual concentration band which is lower than the actual concentration band, after the target is exposed to the chlorine dioxide of the actual concentration band.

The chlorine dioxide of the next actual concentration band may be supplied into a next target space different from the target space, after the chlorine dioxide of the actual concentration band is supplied into the target space. Since the target space should maintain the concentration of the chlorine dioxide when the chlorine dioxide of high concentration satisfying the actual concentration band is supplied into the target space, the target space may be a closed space.

On the other hand, since the concentration of the chlorine dioxide satisfying the next actual concentration band is low, even if the external air is introduced into the next target space according to the access of an operator when the chlorine dioxide is supplied, the target space may maintain the concentration of the chlorine dioxide.

Like this, the target space to which the chlorine dioxide of the actual concentration band is supplied may be different from the next target space to which the chlorine dioxide of the next actual concentration band is supplied. For example, the operator may move the target to the next target space, after the operator exposes the target to the chlorine dioxide satisfying the actual concentration band in the target space. The operator may expose the target to the chlorine dioxide satisfying the next actual concentration band in the next target space.

On the other hand, the chlorine dioxide of the next actual concentration band may be supplied into the target space, after the chlorine dioxide of the actual concentration band is supplied into the target space.

As described above, the target space to which the chlorine dioxide of high concentration is supplied may be a closed space which is blocked from the outside.

In this case, the upper limit concentration of the next actual concentration band may be 0.3 ppm or less, and the lower limit concentration of the next actual concentration band may be 0.01 ppm or more.

After the chlorine dioxide of the actual concentration band is moved, the mixed gas in the target space may be exhausted, and the chlorine dioxide of the next actual concentration band may be transferred.

While the chlorine dioxide of the next actual concentration band is transferred to the next target space, the concentration of the chlorine dioxide in the next target space may be repeatedly increased and decreased in the next actual concentration band.

While the chlorine dioxide of the next actual concentration band is transferred to the target space, the concentration of the chlorine dioxide in the target space may be repeatedly increased and decreased in the next actual concentration band.

The mixed gas having the initial concentration may be firstly provided into the target space, and the mixed gas may be supplied into the target space at the time between an initial providing time of the mixed gas and an exhaust time of the mixed gas at least one time.

The maximum concentration and the minimum concentration of the chlorine dioxide of the mixed gas before the mixed gas is supplied into the target space may be greater than the upper limit concentration and the lower limit concentration of the actual concentration band, respectively.

The reference concentration may be set in a preset acceptable concentration band according to the target exposed to the chlorine dioxide in the target space. The target may be exposed to the chlorine dioxide of the next actual concentration band which is lower than the actual concentration band, after the target is exposed to the chlorine dioxide of the actual concentration band in the target space. In the case of the actual concentration band, the reference concentration may be changed according to the target, and the next actual concentration band may be applied regardless of the target.

The controller 150 may receive, from a timer 210, time information necessary for the apparatus and the method for fumigation using chlorine dioxide such as the point of time of supplying the chlorine dioxide to the target space, the point of time of exhausting the chlorine dioxide from the target space, and the time when the chlorine dioxide is supplied to the target space, in the above description.

The above mentioned agriculture and livestock products may include not only livestock products such as beefs, pork, and chickens, and crops such as fruits, vegetables, and grains but also horticulture crops or medicinal herbs, but the present invention is not limited thereto.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications may be possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

1-40. (canceled)

41. An apparatus for fumigation using chlorine dioxide, the apparatus comprising:
a supply part configured to supply the chlorine dioxide;
a gas sensor configured to sense the chlorine dioxide in a target space into which mixed gas including the chlorine dioxide is injected;
an introduction part configured to introduce dilution gas for diluting the chlorine dioxide;
a mixing part connected to the supply part and the introduction part, and configured to generate the mixed gas by mixing the dilution gas with the chlorine dioxide according to information on a concentration of the chlorine dioxide output from the gas sensor so that the concentration of the chlorine dioxide in the target space may be located within a preset actual concentration band; and
a transfer part connected to the mixing part to transfer the mixed gas to the target space.

42. The apparatus of claim 41, further comprising a controller configured to receive the information from the gas sensor and to output a control signal for adjusting at least one of a flow rate of the dilution gas or a flow rate of the chlorine dioxide so that the concentration of the chlorine dioxide in the target space may be located within the actual concentration band.

43. The apparatus of claim 41, wherein the supply part comprises:
a body including one side in which an electrolyte injection hole into which sodium chlorite is injected and a chlorine dioxide exhaust hole to which the chlorine dioxide is exhausted are formed and the other side, opposite to the one side, in which a surplus gas exhaust hole is formed;
a conductive film provided inside the body;
at least one positive electrode layer which is able to be connected to a current source, and which makes contact with one side of the conductive film; and a negative electrode layer which is able to be connected to the current source, and which makes contact with the other side of the conductive film, opposite to the one side of the conductive film.

44. The apparatus of claim 41, wherein the supply part generates the chlorine dioxide in a gaseous state by passing air bubble through a solvent in which the chlorine dioxide is dissolved.

45. The apparatus of claim 41, wherein the mixing part has a volume smaller than an internal volume of the target space, and a maximum concentration and a minimum concentration of the chlorine dioxide in the mixing part is higher than an upper limit concentration and a lower limit concentration of the actual concentration band, respectively.

46. The apparatus of claim 41, wherein, when the target space is a closed space blocked from an outside of the target space, the introduction part dilutes the chlorine dioxide by introducing an internal air of the target space.

47. The apparatus of claim 41, wherein a lower limit concentration of the actual concentration band is 5 ppm or more, and an upper limit concentration is 300 ppm or less.

48. The apparatus of claim 41, further comprising an air curtain which is connected to the transfer part, and which is installed close to an opening that is formed in the target space and that is able to communicate with the outside of the target space, and which injects the mixed gas into the inside of the target space.

49. The apparatus of claim 41, further comprising a fumigation injecting part which is connected to the transfer part, and which is installed on a ceiling of the target space, and which is configured to inject the mixed gas into the target space through a plurality of injecting holes which are spaced apart from each other.

50. The apparatus of claim 41, wherein a reference concentration is set within a preset acceptable concentration band according to a target exposed to the chlorine dioxide in the target space, an upper limit concentration of the actual concentration band is set according to the reference concentration and an upper limit offset, a lower limit concentration of the actual concentration band is set according to the reference concentration and a lower limit offset, and the upper limit concentration and the lower limit concentration are values located within the acceptable concentration band.

51. The apparatus of claim 41, wherein a concentration of the chlorine dioxide in the target space is located within the actual concentration band, and is increased and decreased repeatedly for an exposure time when a target is exposed to the chlorine dioxide.

52. The apparatus of claim 41, wherein a target exposed to the chlorine dioxide is exposed to a next actual concentration band which is lower than the actual concentration band, after being exposed to the chlorine dioxide of the actual concentration band in the target space.

53. The apparatus of claim 41, wherein the mixed gas having an initial concentration is firstly provided to the target space, and the mixed gas is supplied into the target space at least one time during a time between an initial providing time of the mixed gas and an exhaust time of the mixed gas.

54. The apparatus of claim 41, wherein a maximum concentration and a minimum concentration of the chlorine dioxide of the mixed gas before the mixed gas is supplied into the target space is greater than an upper limit concentration and a lower limit concentration of the actual concentration band, respectively.

55. The apparatus of claim 41, wherein a reference concentration is set in a preset acceptable concentration band according to a target exposed to the chlorine dioxide in the target space, the target is exposed to the chlorine dioxide of a next actual concentration band which is lower than the actual concentration band, after the target is exposed to the chlorine dioxide of the actual concentration band in the target space, and the reference concentration is able to be changed according to the target in a case of the actual concentration band and the next actual concentration band is applied regardless of the target.

56. A method for fumigation using chlorine dioxide, the method comprising:
supplying chlorine dioxide;
sensing the chlorine dioxide in a target space;
generating mixed gas by mixing dilution gas with the supplied chlorine dioxide according to information on a concentration of the sensed chlorine dioxide so that the concentration of the chlorine dioxide in the target space may be located within a preset actual concentration band; and
transferring the mixed gas to the target space.

57. The method of claim 56, wherein at least one of a flow rate of the dilution gas or a flow rate of the chlorine dioxide is adjusted according to the information so that the concentration of the chlorine dioxide in the target space may be located within the actual concentration band.

58. The method of claim 56, wherein a lower limit concentration of the actual concentration band is 5 ppm or more and an upper limit concentration is 300 ppm or less.

59. The method of claim 56, wherein a reference concentration is set within a preset acceptable concentration band according to a target exposed to the chlorine dioxide in the target space, an upper limit concentration of the actual concentration band is set according to the reference concentration and an upper limit offset, a lower limit concentration of the actual concentration band is set according to the reference concentration and a lower limit offset, and the upper limit concentration and the lower limit concentration are a value located within the acceptable concentration band.

60. The method of claim 56, wherein a concentration of the chlorine dioxide in the target space is located within the actual concentration band, and is increased and decreased repeatedly for an exposure time when a target is exposed to the chlorine dioxide.

61. The method of claim 56, wherein a target exposed to the chlorine dioxide is exposed to a next actual concentration band which is lower than the actual concentration band, after being exposed to the chlorine dioxide of the actual concentration band.

62. The method of claim 56, wherein the target space is a closed space blocked from an outside of the target space.

63. The method of claim 56, wherein the mixed gas having an initial concentration is firstly provided to the target space, and the mixed gas is supplied into the target space during a time between an initial providing time of the mixed gas and an exhaust time of the mixed gas, at least one time.

64. The method of claim 56, wherein a maximum concentration and a minimum concentration of the chlorine dioxide of the mixed gas before the mixed gas is supplied into the target space is greater than an upper limit concentration and a lower limit concentration of the actual concentration band, respectively.

65. The method of claim 56, wherein a reference concentration is set in a preset acceptable concentration band according to a target exposed to the chlorine dioxide in the target space, the target is exposed to the chlorine dioxide of a next actual concentration band which is lower than the actual concentration band, after the target is exposed to the chlorine dioxide of the actual concentration band in the target space, and the reference concentration is able to be changed according to the target in a case of the actual concentration band and the next actual concentration band is applied regardless of the target.
